(19) United States
(12) Patent Application Publication
(10) Pub. No.: US 2026/0315773 A1
(43) Pub. Date: Oct. 15, 2026

(54) RAPID EXCHANGE BLOOD PUMP WITH STRAIGHT GUIDEWIRE PATH THROUGH PUMP HOUSING

(71) Applicant: CARDIOVASCULAR SYSTEMS, INC., St. Paul, MN (US)

(72) Inventors: Sourav K. Bhunia, Shoreview, MN (US); Tristan Van de Moortele, Minneapolis, MN (US); Joseph P. Higgins, Wayzata, MN (US)

(21) Appl. No.: 19/106,491

(22) PCT Filed: Aug. 25, 2023
(86) PCT No.: PCT/US2023/031146

§ 371 Date: Feb. 25, 2025

Related U.S. Application Data
(60) Provisional application No. 63/374,437, filed on Sep. 2, 2022.

(51) Int. Cl. A61M 60/13; A61M 60/174; A61M 60/216; A61M 60/806; A61M 60/81; A61M 60/825; A61M 60/865

(52) U.S. Cl. CPC A61M 60/13; A61M 60/174; A61M 60/216; A61M 60/806; A61M 60/81; A61M 60/825; A61M 60/865; A61M 2205/04; A61M 2205/103; A61M 2210/125

(57) ABSTRACT

A percutaneous ventricular assist devices, systems and methods are disclosed and provide for a linear guidewire path that does not increase crossing profile and allows the guidewire to take a substantially linear path through the device. The device comprises a rotatable impeller with blades formed from an impeller hub. Notch(es) or apertur(es) are provided through the impeller hub or blade(s) to enable passage of a guidewire therethrough. The impeller blade is rotated to align the notch(es) or aperture(es) with a proximally located stationary guidewire path through a proximal bearing region and/or a proximal housing region. Once the non-stationary guidewire path element is aligned with the stationary guidewire path element, the guidewire may translated along the aligned guidewire path.

### CROSS-REFERENCE TO RELATED APPLICATIONS

This application claims priority to provisional application No. 63/374,437, filed Sep. 2, 2022 and titled RAPID EXCHANGE BLOOD PUMP WITH STRAIGHT GUIDEWIRE PATH THROUGH PUMP HOUSING, the entire contents of which are incorporated herein by reference.

### FIELD OF THE INVENTION

The invention relates to an intravascular rotational blood pump device with an impeller located within a housing and a guidewire path that is straight and located within the blood pump's housing wherein a guidewire disposed along the guidewire path does not increase the outer diameter of the blood pump housing.

### BACKGROUND

With reference to FIG. 1 shown below, the human heart comprises four chambers and four heart valves that assist in the forward (antegrade) flow of blood through the heart. The chambers include the left atrium, left ventricle, right atrium and left ventricle. The four heart valves include the mitral valve, the tricuspid valve, the aortic valve and the pulmonary valve.

The mitral valve is located between the left atrium and left ventricle and helps control the flow of blood from the left atrium to the left ventricle by acting as a one-way valve to prevent backflow into the left atrium. Similarly, the tricuspid valve is located between the right atrium and the right ventricle, while the aortic valve and the pulmonary valve are semilunar valves located in arteries flowing blood away from the heart. The valves are all one-way valves, with leaflets that open to allow forward (antegrade) blood flow. The normally functioning valve leaflets close under the pressure exerted by reverse blood to prevent backflow (retrograde) of the blood.

Thus, as illustrated, the general blood flow comprises deoxygenated blood returning from the body where it is received by the right atrium via the superior and inferior vena cava and is, in turn, pumped into the right ventricle, a process controlled by the tricuspid valve. The right ventricle functions to pump the deoxygenated blood to the lungs via the pulmonary arteries, where the blood is reoxygenated and returned to the left atrium via the pulmonary veins.

Heart disease is a health problem with a high mortality rate. The use of temporary mechanical blood pump devices are used on an increasingly frequent basis to provide short-term acute support during surgery or as temporary bridging support to help a patient survive a crisis. These temporary blood pumps have developed and evolved over the years to supplement the pumping action of the heart on a short-term basis and supplement blood flow as either left or right ventricular assist devices, with the left ventricular assist device (“LVAD”) currently the most commonly used device.

Known temporary LVAD devices generally are delivered percutaneously, e.g., through the femoral artery, to locate or position the LVAD inlet in the patient's left ventricle and the outlet in the patient's ascending aorta with the body of the device disposed across the aortic valve. As the skilled artisan will understand, an incision may be made below the patient's groin to enable access to the patient's femoral artery. The physician may then translate guide wire, followed by a catheter or delivery sheath, through the femoral artery and descending aorta until reaching the ascending aorta. The LVAD with attached rotational drive shaft may then be translated through the delivery catheter or sheath lumen, leaving a proximal end of the drive shaft exposed outside of the patient and coupled with a prime mover such as an electric motor or the equivalent for rotating and controlling the rotational speed of the drive shaft and associated LVAD impeller.

Temporary axial flow blood pumps consist generally of two types: (1) those that are powered by a motor integrated into the device that is connected with the pump's impeller (see U.S. Pat. Nos. 5,147,388 and 5,275,580) ; and (2) those that are powered by an external motor that provides rotational torque to a drive shaft which is, in turn, connected to the pump's impeller (see U.S. Pat. No. 4,625,712 to Wampler and U.S. Pat. No. 5,112,349 to Summers, each hereby incorporated by reference in their entirety).

Known temporary ventricle assist devices (“VAD”), including LVAD and RVAD (right ventricular assist) devices of the present invention comprises an external motor, and may generally comprise the following elements mounted within a housing, listed in order from the inflow end to the outflow end: an inflow aperture(s); a stationary inducer, also known as a flow straightener; a rotational impeller; and a stationary diffuser and/or outflow structure; and an outflow aperture(s).

FIG. 2A illustrates an exemplary blood pump procedure which may be an LVAD or an RVAD procedure. As shown, the handle or controller comprises an electric motor therein which is rotatably connected with a rotational drive shaft extending distally away from the electric motor and which is disposed within a sheath or catheter.

FIG. 3 illustrates an exemplary blood pump cannula housing structure positioned with inflow apertures located within a patient's left atrium and outflow apertures through the blood pump cannula housing located on the downstream side of the patient's aortic valve. An impeller (not shown) is rotated by actuation of the electric motor, which rotates the drive shaft and the impeller, causing blood to flow into the inflow apertures and out through the outflow apertures as shown.

Rapid exchange percutaneous transcatheter ventricular-assist device (pVAD), including LVAD and RVAD devices, has been taught in the art. However, the known rapid exchange pVAD devices increase either the cross-sectional dimension of the pVAD profile during insertion into a patient (Hastie et al., U.S. Pat. No. 8,814,776) or the actual diameter of the pVAD (Aboul-Hosn et al., U.S. Pat. No. 7,731,675), making the size of the incision necessary for implantation larger than the minimum necessary without the rapid exchange feature. Typically, the cross-section of a pVAD varies along its length. The largest cross-section of the device while being implanted determines the size of the incision and the difficulty or ease of traversing tortuous vasculature.

Accordingly, pVADs are typically designed to minimize the size of its cross-section necessary to accomplish its primary function of delivering a certain flow rate of blood. The cross-sectional diameter, also known as crossing profile, is determined primarily by the impeller, the cannula or housing surrounding around the impeller which typically has the largest outer diameter in pVAD devices.

Hastie et al. in U.S. Pat. No. 8,814,776(“'776 ”) discloses a removable catheter with a lumen for guidewire that is to be kept in place during the pVAD placement in a patient. This catheter is to be removed after the pVAD is placed in a patient. However, during the pVAD placement, the largest cross-sectional dimension of the pVAD is equal to the sum of the removable catheter outer diameter and the outer diameter of the cannula in the region of the impeller and motor in the region of the motor. This arrangement places the guidewire outside of a portion of the LVAD housing, which is the portion of the device having the largest diameter. The result of this prior art device is that the outer diameter along at least a portion of the LVAD housing includes the outer diameter of the LVAD housing plus the added diameter of the guidewire. As a result, the effective outer diameter of a device according to the '776 patent is greater than the outer diameter of LVAD housing alone. In addition, the '776 patent requires the guidewire to curve during insertion and/or removal from the pVAD device, thereby increasing friction and difficulty in positioning the guidewire along the non-linear guidewire path.

Aboul-Hosn in U.S. Pat. No. 7,731,675 (“'675”) teaches a rapid exchange or “side-rigger” design that includes a guidewire lumen within the overall cross-sectional profile of a pVAD in some sections, but not in the region of its largest cross-section. The largest cross-sectional dimension of the pVAD in the region of the impeller is equal to the sum of the outer diameter of the cannula in the region and that of the guidewire.

In addition, the '675 patent teaches that the guidewire is disposed within a wall of the pVAD housing, requiring an inward bulge in the pVAD housing, thus creating a non-circular fluid flow passageway.

Among other things, the inventors of the presently disclosed inventive embodiments discovered that the non-circular fluid flow passageway creates a significant hydraulic loss.

Further, the '675 patent teaches an alternative embodiment wherein the guidewire is disposed within a wall of the pVAD housing, with a circular fluid flow passageway, but wherein the presence of the guidewire requires an outward bulge in the pVAD housing and resulting increase outer diameter.

Thus, there is a need for a rapid exchange pVAD that does not require increasing diameter to accommodate a guidewire, preserves the full circular flow path through the device's housing and provides a linear, non-curving, pathway through the device for the guidewire.

Accordingly, embodiments of the present invention address, inter alia, the issues described above.

### BRIEF DESCRIPTION OF THE DRAWINGS

The inventions will be further described with reference to the accompanying drawings which illustrate exemplary embodiments and are not limiting.

FIG. 1 is a cross-sectional view of the human heart.

FIG. 2A illustrates a physician performing an exemplary intravascular procedure.

FIG. 2B illustrates the handle with integrated motor of FIG. 2A.

FIG. 3 illustrates a cross-sectional view of the human heart with a blood pump positioned therein.

FIG. 4 is a perspective, partial cutaway view of one embodiment of the present disclosure.

FIG. 5 is an end view of a known impeller.

FIG. 6A is a cross-sectional view of one embodiment of the present disclosure.

FIG. 6B is a side, cutaway view of the embodiment of FIG. 6A.

FIG. 7 is an end view of a prior art impeller.

FIG. 8 is an end view of one embodiment of an impeller of the present disclosure.

FIG. 9 is a perspective cutaway view of one embodiment of the present disclosure.

FIG. 10 is a perspective cutaway view of one embodiment of the present disclosure.

FIG. 11 is a perspective cutaway view of the embodiment of FIG. 10.

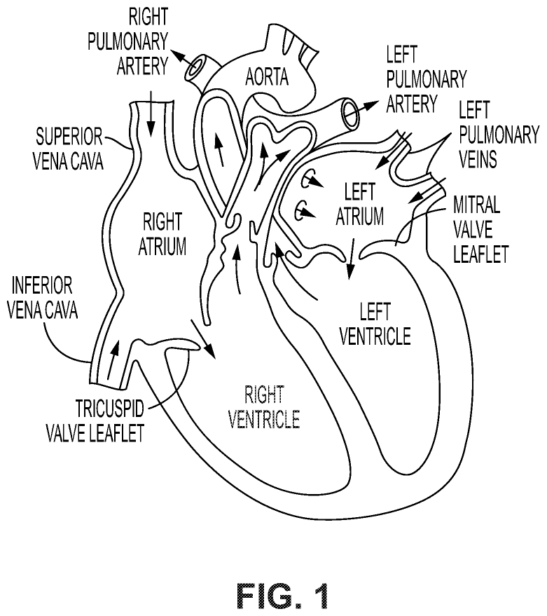

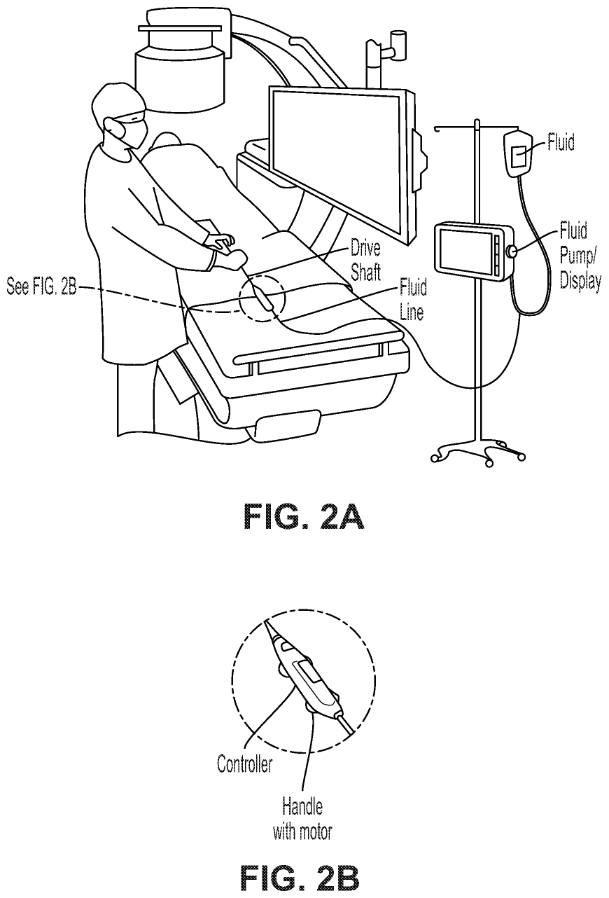

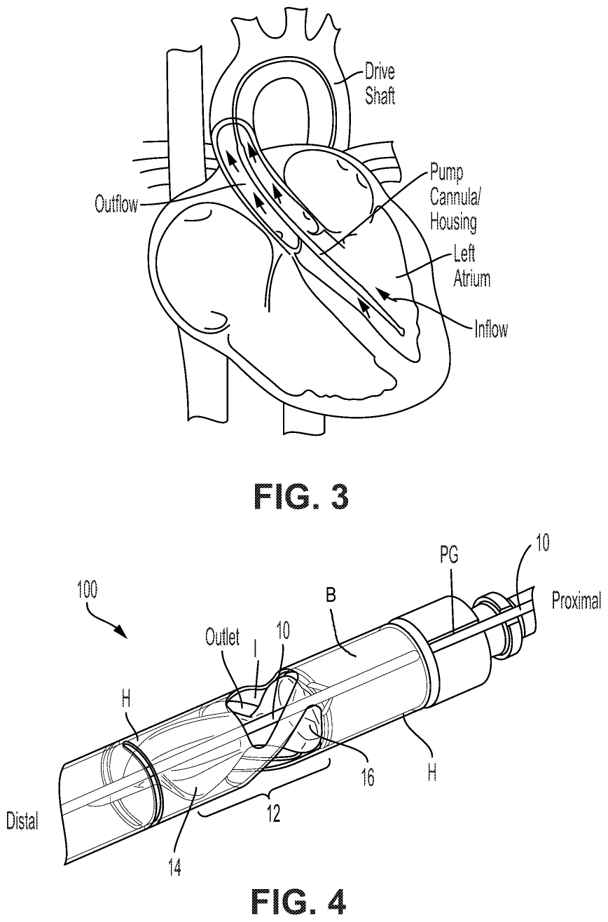

### DETAILED DESCRIPTION

While the invention is amenable to various modifications and alternative forms, specifics thereof are shown by way of example in the drawings and described in detail herein. It should be understood, however, that the intention is not to limit the invention to the particular embodiments described. On the contrary, the intention is to cover all modifications, equivalents, and alternatives falling within the spirit and scope of the invention.

A rapid exchange blood pump, e.g., a percutaneous ventricular assist device or “pVAD” is described wherein the guidewire lumen or pathway defined therein, does not increase the largest cross-section of the pVAD. In other words, the outer diameter of the cannula in the region of the impeller is not affected or enlarged by the presence of either the guidewire pathway or the guidewire itself. In addition, a substantially linear guidewire path is provided through the housing of the LVAD and, specifically, through the interior space defined within the housing of the pVAD within the region of the impeller. Moreover, the highly desirable circular flow path through the pVAD housing is preserved.

As discussed above, for a pVAD with an external, non-implanted, motor, the cannula outer diameter in the region of the impeller becomes the largest diameter of the device and thus determines, among other things, the size of the incision necessary for the insertion or implantation and the difficulty in traversing vasculature.

One embodiment of the present invention is shown in FIG. 4. As shown, a portion of an exemplary pVAD device, or blood pump 100 is illustrated. A guidewire 10 is disposed along a substantially linear path along a portion of the housing and through the interior of the housing of the pVAD. A proximal portion of the guidewire path is formed along or defined by a linear groove or channel PG defined by the housing H on at least the proximal side of the impeller 12 and outlets. During loading, as will be discussed further below, the guidewire 10 extends within an interior space I defined by housing H in the region of the impeller 12 and exits the interior space I guided within the linear groove or channel PG on the proximal side of the housing H.

As described further herein, the guidewire 12 extends through a portion of the blades 14 of the impeller 12 to maintain a substantially linear guidewire path. More specifically, the guidewire 10 extends through at least one blade 14 and, in some embodiments, through the hub 16 to which the blade(s) 14 are attached. This allows a substantially linear guidewire path to be formed through the impeller 12 structure and without engaging the housing H surrounding the impeller 12. Thus, when the guidewire 10 is disposed along and/or through the impeller 12, the guidewire 10 is not located within the housing H walls, but is located within an interior space I defined by the housing H. Accordingly, the guidewire path defined thereby is substantially linear and substantially aligned with a longitudinal axis that is radially offset from a central longitudinal axis through the housing H. In this embodiment, the guidewire 10 may extend distally through the interior space I defined by the housing. In other embodiments, the guidewire 10 may exit the interior space I at a point that is distal to the impeller 12.

Thus, the hub 16 and/or blade(s) 14 of the stationary impeller 12 and, in some embodiments, the housing H proximal to the impeller 12 may define the guidewire path to create an unobstructed guidewire path that is substantially straight or linear when the grooved or channeled PG portions of the guidewire path are aligned by rotating the impeller 12. The resulting guidewire path is preferably large enough to accommodate a guidewire 10 that is typically 0.020″ or smaller in diameter, though larger guidewire sizes may also be accommodated.

The shapes of the openings created through provision of the grooves or channels PG of the stationary and non-stationary portions of the guidewire path do not have to be perfectly aligned in a longitudinal or linear orientation, but they must substantially overlap or substantially align in at least at one orientation of the impeller 12 as it rotates around its axis. It is preferable to provide a substantially linear alignment between the grooves or channels PG and the openings defined by the blade 14 and/or hub.

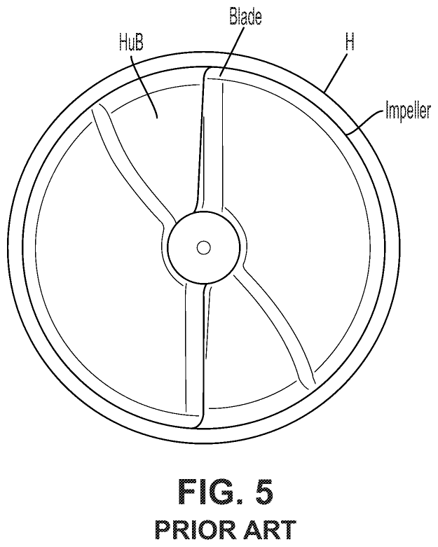

The traversal of the guidewire 10 through or along the impeller 12 as in FIG. 4 is not possible with known impeller structures. For example, a prior art impeller is shown in FIG. 5. There, the blades extend in a fully circumferential manner providing a small clearance between the blades and the housing H. The small clearance is insufficient to allow traversal of a guidewire through or along the impeller.

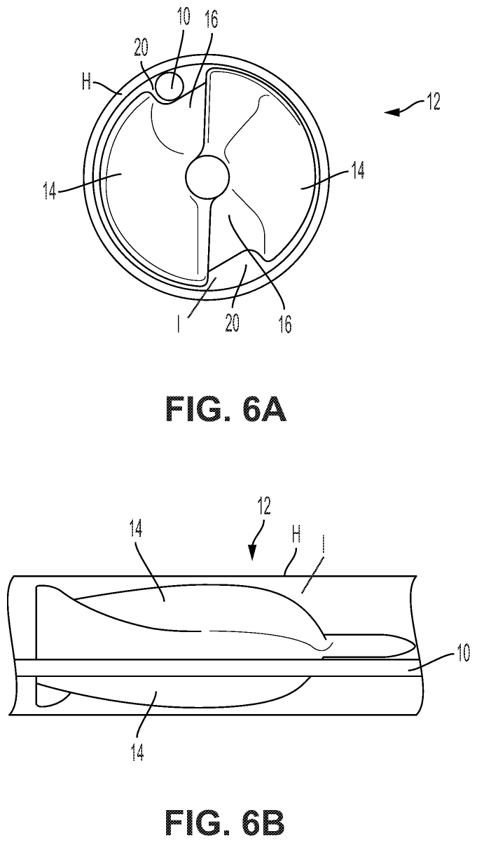

The traversal of the guidewire 10 through or along the impeller 12 shown in FIG. 4 is further illustrated in FIGS. 6A and 6B. Thus, viewing the stationary portion of the guidewire path axially through a cutaway view of the housing H, the defined guidewire path provides a substantially linear or substantially straight path for the guidewire 10 to pass through the impeller 12 structure located within the interior space I defined by the housing H. The guidewire 10 passes through a clearance defined between the housing H and the blade 14 and/or hub 16 of the impeller 12. As a result, there is no bending of the guidewire 10 and, therefore, no resulting resistance or friction created by the guidewire as it passes through the impeller 12 and into the more proximal bearing housing H (not pictured). In addition, the blood flow path remains circular which is most efficient.

FIG. 6A provides an exemplary embodiment of the modification of the impeller hub 16 and/or blade(s) 14 to define the portion of the guidewire path that extends therealong and therethrough. Accordingly, a notch or aperture 20 may be provided along at least a portion of the outer blade 14 and/or hub 16 as best shown in FIG. 6A to define the openings discussed above that allow the guidewire 10 to be disposed in a substantially linear or straight configuration along the resultant non-stationary guidewire path. The notch or aperture 20 is sized to accommodate a guidewire 10 therethrough or therealong.

FIG. 6B provides a side view of the impeller 12 of FIG. 6A illustrating guidewire 10 located along the impeller 12 and the housing H. The substantially straight traversal of the guidewire 10 along, or through, the impeller within the interior space I defined by the housing H is made possible by the notch(es) or aperture(s) 20.

Embodiments of the present invention may define one or more one guidewire paths along the non-stationary component of the guidewire path. The number of guidewire paths facilitated by the impeller notch(es) or aperture(s) 20 of FIGS. 6A and 6B within the non-stationary component of the guidewire path may be equal to the number of impeller blades 14 that comprise a notch or aperture 20, or the number of notches or apertures 20 defined by the hub 16. Thus, there may be one guidewire path, or more than one guidewire path, formed or defined by at least the impeller notch(es) or aperture(s) 20.

In addition, there may be one, or more than one, defined guidewire path in the stationary component of the guidewire path. The defined guidewire path(s) in the stationary component of the guidewire path may be equal to the number of impeller blades 14. Alternatively, the defined guidewire path(s) in the stationary component may be equal to the number of notches or apertures 20 in outer region or portion of the blades 14 and/or hub 16. As the artisan will now recognize, turning the impeller 12 to substantially align the stationary and non-stationary components of the guidewire path allows the guidewire 10 to pass along, or occupy, the guidewire path in a substantially linear manner.

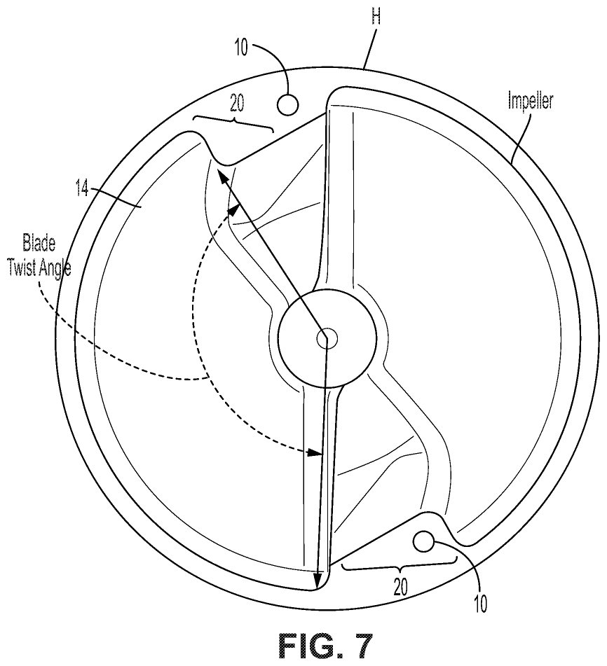

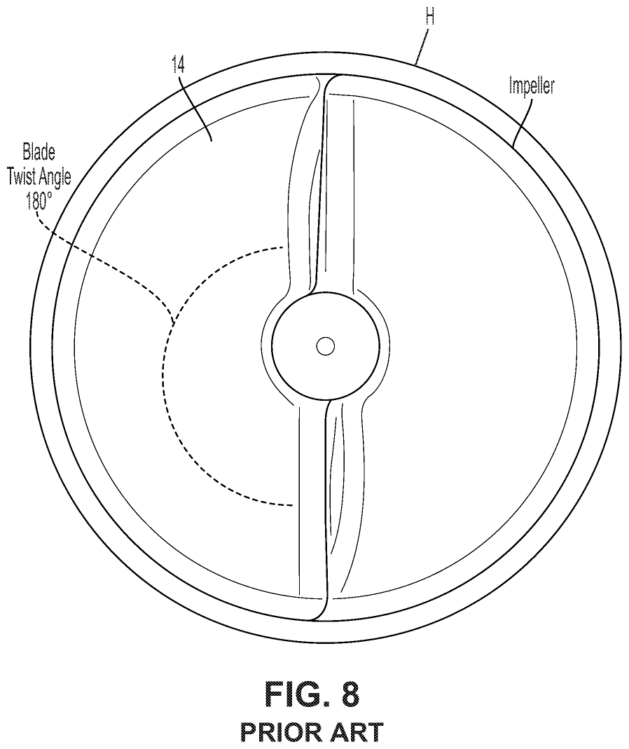

Additionally, in certain embodiments, the impeller 12 may comprise a “blade twist” that is less than 180 degrees. For example, FIG. 7 illustrates one embodiment comprising a blade twist angle that is less than 180 degrees, e.g., approximately 140 degrees, and which allows for creating the notch(es) or aperture(s) 20 described above and defining a guidewire path through the non-stationary component of the guidewire path.

This value represents how much the blade 14 rotates from the start to the end of the impeller 12 axially or in the longitudinal direction. In this embodiment, the blade twist allows room for the guidewire 10 to pass along the defined guidewire path while remaining substantially linear. As shown in FIG. 7, an exemplary blade twist may comprise approximately 140 degrees, though any blade twist that is less than 180 degrees is within the scope of the invention so long as the guidewire 10 is enabled to pass through the notch or aperture 20.

FIG. 8 illustrates a prior art impeller, with a blade twist angle of approximately 180 degrees, indicated by the dashed lines, leaving no room for a guidewire path therethrough or therealong.

Thus, at least a portion of the more proximal stationary guidewire path described above may comprise a funnel F defined along a bearing structure B that is operationally connected with a proximal portion of the impeller 12. Thus, the funnel F is located proximal to the impeller 12, and in fluid communication with the interior of the pVAD housing and substantially align-able with the non-stationary more distal portion of the guidewire path as described above.

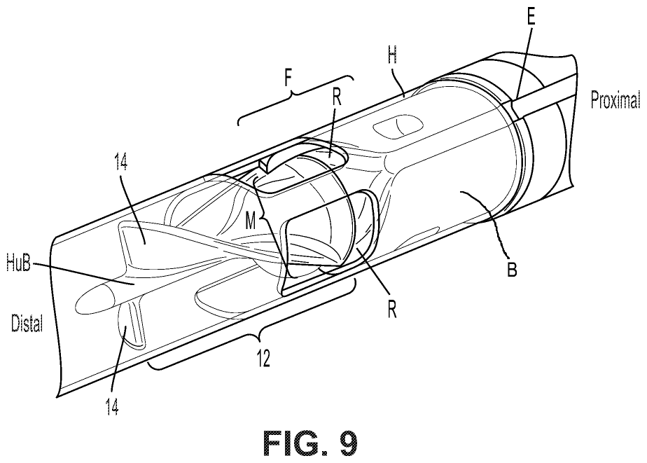

As shown in FIGS. 9-11, the larger diameter, more distal mouth M of the funnel F creates a radiused or tapering ramp R to between the straight stationary guidewire path and the non-stationary guidewire path to aid in guiding the guidewire 10 loading from the distal end of the pVAD. As the guidewire 10 is loaded along the guidewire path through the more distal non-stationary guidewire path it will next encounter the mouth M (wider, distal portion) of the funnel such that the guidewire's most proximal tip will ride or move along the wall W of the funnel toward the straight or linear stationary portion of the guidewire path.

As noted supra, the housing H may comprise in certain embodiments a stationary guidewire path component comprising a proximal housing groove PG as shown in FIGS. 4 and 9-11 to accommodate receiving at least a portion of the guidewire 10 and to allow translation and/or rotation of the guidewire 10 therealong and therethrough. The proximal housing groove PG is in operative communication with the funnel F such that as the guidewire 10 is guided proximally up the tapering ramp F of the funnel F, the guidewire 10 is further guided into the proximal housing groove PG. As best shown in FIGS. 9-11, the proximal housing groove PG may terminate at an exit aperture E to allow the guidewire 10 to exit from the interior space I defined by housing H in the region of the impeller 12.

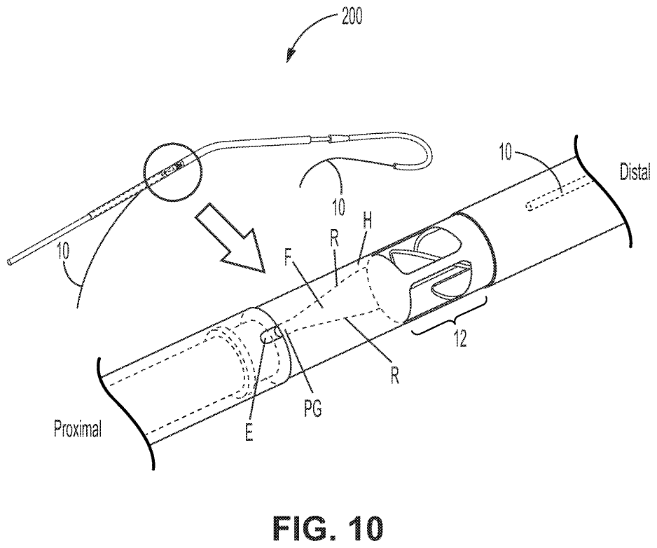

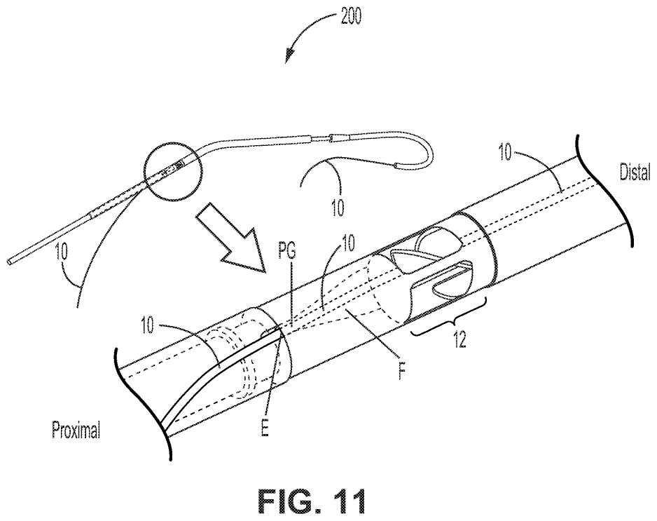

FIGS. 10 and 11 illustrate an exemplary guidewire 10 loading method. There, an embodiment of a pVAD device 200 is shown and comprising an impeller 12 comprising at least one notch or aperture as discussed above to form at least one non-stationary guidewire path. The impeller is disposed within a housing H. The guidewire 10 is first loaded into the distal end of the pVAD device 200 and translated in a proximal direction toward the impeller 12. The impeller 12 (non-stationary component of the guidewire path(s)) is rotated so that the at least one notch or aperture 20 (not shown but as described above) is substantially aligned with the proximal groove PG. When substantial alignment is achieved between at least one of the notches or apertures 20, the guidewire 10 may be advanced through or along the impeller 12 by translating the guidewire 10 through the aligned notch or aperture 20 and toward the funnel ramp R. The funnel ramp R guides the proximally advancing guidewire 10 toward the aligned proximal groove PG and out of the interior space I of the housing through the exit aperture E.

The description of the invention and is as set forth herein is illustrative and is not intended to limit the scope of the invention. Features of various embodiments may be combined with other embodiments within the contemplation of this invention. Variations and modifications of the embodiments disclosed herein are possible and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

1. A blood pump comprising:
a housing defining an interior space;
a rotatable impeller disposed within the interior space and comprising an impeller hub and one or more impeller blades disposed along the impeller hub;
a stationary bearing region to which a proximal end of the impeller is rotationally operationally engaged;
a stationary guidewire path defined along at least a portion of the stationary bearing region, the stationary guidewire path located within the interior space defined by the housing; and
a non-stationary guidewire path extending through the interior space of the housing along the impeller,
wherein the stationary and non-stationary guidewire paths are configured to be substantially align-able and substantially linear in a longitudinal direction when the non-stationary guidewire path is moved into alignment with the stationary guidewire path.

2. The blood pump of claim 1, wherein the stationary guidewire path comprises a proximal groove or channel defined within the interior space defined by the housing and disposed along a longitudinal axis, the proximal groove or channel configured to receive a guidewire at least partially therein; and
wherein the proximal groove or channel is located proximally of the rotatable impeller.

3. (canceled)

4. The blood pump of claim 2, wherein at least a portion of the proximal groove or channel is at least partially defined by the stationary bearing region.

5. The blood pump of claim 1, wherein the non-stationary guidewire path comprises at least one notch or aperture defined by at least one of the one or more impeller blades, the at least one notch or aperture configured to receive a guidewire therethrough.

6. The blood pump of claim 1, wherein the non-stationary guidewire path comprises at least one notch or aperture defined by the impeller hub, the at least one notch or aperture configured to receive a guidewire therethrough.

7. The blood pump of claim 1, wherein the non-stationary guidewire path comprises at least one notch or aperture defined by at least one of the one or more impeller blades and/or the impeller hub, the at least one notch or aperture configured to receive a guidewire therethrough.

8. The blood pump of claim 1, wherein the non-stationary guidewire path comprises an impeller with the one or more blades comprising a twist angle that is less than 180 degrees.

9. The blood pump of claim 8, wherein the non-stationary guidewire path comprises an impeller with the one or more blades comprising a twist angle that is greater than about 140 degrees and less than about 180 degrees.

10. The blood pump of claim 8, wherein the non-stationary guidewire path comprises an impeller with the one or more blades comprising a twist angle that is about 140 degrees.

11. The blood pump of claim 1, wherein the stationary guidewire path comprises a portion of the stationary bearing region defining a funnel comprising a mouth of largest diameter at a distal end and a ramp that tapers down in the proximal direction.

12. The blood pump of claim 2, wherein the stationary guidewire path comprises a portion of the stationary bearing region defining a funnel comprising a mouth of largest diameter at a distal end and a ramp that tapers down in the proximal direction, and
wherein a proximal end of the funnel aligns with the proximal groove or channel, the proximal groove or channel configured to receive a guidewire therein.

13. The blood pump of claim 11, wherein the ramp tapers symmetrically relative to each other in the proximal direction.

14. The blood pump of claim 11, wherein the ramp tapers asymmetrically relative to each other in the proximal direction.

15. A method for providing a substantially linear guidewire path through a percutaneous ventricular assist device comprising an impeller with one or more blades defined along an impeller hub, a stationary bearing region proximal to the impeller and a housing region, the method comprising:
providing at least one notch or aperture along a portion of the one or more impeller blades to allow passage of a guidewire therethrough;
defining a stationary guidewire path along at least a portion of the stationary bearing region;
rotating the impeller to align the at least one notch or aperture with the stationary guidewire path; and
translating the guidewire through the stationary guidewire path and the aligned notch or aperture.

16. The method of claim 15, wherein the stationary guidewire path comprises defining a proximal groove or channel along the stationary bearing region; and
wherein the stationary guidewire path comprises providing a funnel comprising a proximally tapering ramp, the funnel defined by the stationary bearing region.

17. (canceled)

18. The method of claim 16, wherein the stationary guidewire path comprises providing a proximal groove or channel along the stationary bearing region proximal to the impeller that is aligned with a proximal portion of the funnel.

19. A method for providing a substantially linear guidewire path through a percutaneous ventricular assist device comprising an impeller with one or more blades defined along an impeller hub, a stationary bearing region proximal to the impeller and a housing region, the method comprising:
providing the impeller with blades comprising a twist angle that is less than 180 degrees to comprise a non-stationary guidewire path;
providing a stationary guidewire path along the stationary bearing region, the stationary guidewire path at least partly within an interior space I defined by the housing region H, the stationary guidewire path located proximal to the impeller;
rotating the impeller to align the non-stationary guidewire path with the stationary guidewire path; and
translating the guidewire through the aligned non-stationary guidewire path and the stationary guidewire path.

20. The method of claim 19, wherein the stationary guidewire path comprises providing a proximal groove or channel along the housing region proximal to the impeller.

21. The method of claim 19, wherein the stationary guidewire path comprises providing a funnel comprising a proximally tapering ramp defined by the stationary bearing region, wherein the funnel is surrounded by the housing region.

22. The method of claim 20, wherein the proximal groove or channel is substantially aligned with a proximal portion of the funnel.
